(19) United States
(12) Patent Application Publication
(10) Pub. No.: US 2022/0317333 A1
(43) Pub. Date: Oct. 6, 2022

(54) METHOD FOR ANALYZING THE CONSTITUTIVE RELATIONSHIP OF CONFINED ROCK EXPANSION

(71) Applicant: Research Institute of Highway Ministry of Transport, Beijing (CN)

(72) Inventors: Chongbang XU, Beijing (CN); Xiaojing GAO, Beijing (CN); Youlin QIN, Beijing (CN); Xuan ZHANG, Beijing (CN)

(21) Appl. No.: 17/712,846

(22) Filed: Apr. 4, 2022

(30) Foreign Application Priority Data
Apr. 2, 2021 (CN) 202110360327.X

(51) Int. Cl. G01V 99/00; G06F 30/20

(52) U.S. Cl. CPC G01V 99/005; G06F 30/20; G06F 2111/10

(57) ABSTRACT

The invention discloses a method for analyzing the constitutive relationship of confined rock expansion, including: (1) determining parameters that affect rock expansive properties, wherein the parameters comprise category, density, and water content; (2) establishing an expression of the relationship between the parameters and rock expansive capacity; (3) establishing an expression of the relationship among the rock expansive capacity, rock expansive stress and rock expansive strain; and (4) determining an expression of the relationship among the parameters, the rock expansive stress and the rock expansive strain according to the rock expansive capacity. The present invention introduces parameters comprising category, density, and water content that affect the rock expansive properties into the analysis of rock expansive stress and rock expansive strain, which can truly reflect the variation of rock expansive stress and rock expansive strain, and can achieve quantized calculation of the rock expansive state.

### TECHNICAL FIELD

The present invention relates to the field of methods for analyzing rock engineering mechanics, and in particular, to a method for analyzing the constitutive relationship of confined rock expansion.

### BACKGROUND

Rock expansion is an inherent mechanical property of the rock, and when encountering water, the expansive rock will undergo a physicochemical reaction to generate volume expansion. Since the process mechanically acts quite pronounced, the encountering mechanics is an important content in the field of engineering mechanics. In the rock expansive process, expansive stress is in a negative correlation with expansive strain, which does not conform to the classical elastic mechanics intrinsic relationship. Currently, how to test the maximum of the expansive stress, how to test the maximum of the free expansion rate, and how to regressively analyze the test data are usually the focus of attention. However, these theories are difficult to reflect the relationship among rock water content, rock expansive stress and rock expansive strain at the same time.

Therefore, it is necessary to develop a new method for analyzing the constitutive relationship of confined rock expansion, which has not only high research value, but also good economic benefit and industrial application potential.

### SUMMARY

The present invention provides a method for analyzing the constitutive relationship of confined rock expansion, which can solve the technical problem in the prior art that rock water content, rock expansive stress and rock expansive strain cannot be collectively expressed to calculate and identify the rock expansive state.

According to an aspect of the present invention, there is provided a method for analyzing the constitutive relationship of confined rock expansion, comprising:

(1) determining parameters that affect rock expansive properties, wherein the parameters comprise category, density, and water content;

(2) establishing an expression of the relationship between the parameters and rock expansive capacity;

(3) establishing an expression of the relationship among the rock expansive capacity, rock expansive stress and rock expansive strain; and

(4) determining an expression of the relationship among the parameters, the rock expansive stress and the rock expansive strain according to the rock expansive capacity.

Preferably, wherein in step (2), the relationship between the parameters and the rock expansive capacity is as follows:

$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} ;$$

wherein, Qₛ is the rock expansive capacity, kᵣ is rock expansive active coefficient, m·N/kg, ρ_{d} is rock density, kg/m³, w is water content in rock expansive state, w₀ is water content in rock expansive initial state, wₘₐₓ is water content in rock expansion reaching limit state, wᵥ is water content at the point when the water absorption rate changes from rapid increase to slow increase in the rock expansive process.

Preferably, wherein in step (3), the relationship among the rock expansive capacity, the rock expansive stress and the rock expansive strain is as follows:

Qₛ=Q_{σ}+Q_{ε};

wherein, Qₛ is the rock expansive capacity, Q_{σ} is rock expansive capacity corresponding to the rock expansive stress and Q_{ε}is rock expansive capacity corresponding to the rock expansive strain.

The relationship between the rock expansive stress Q_{σ} and its corresponding rock expansive stress σₚ is as follows:

Q_{σ}=βₚ;

wherein, β is conversion coefficient of the rock expansive stress.

The relationship between the rock expansive strain εₚ and its corresponding rock expansive capacity Q_{ε} is as follows:

Q_{ε}=ωεₚ^{0.5};

wherein, ω is conversion coefficient of the rock expansive strain, kPa.

That is:

Qₛ=βσₚ+ωεₚ^{0.5}

Preferably, wherein in step (4), the rock expansive capacity is:

$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} ;$$

$${Q}_{s} = {Q}_{σ} + {Q}_{ε} = β {σ}_{p} + {ωε}_{p}^{0.5}$$

The relationship among the parameters, the rock expansive stress and the rock expansive strain is as follows:

$${σ}_{p} = \frac{1}{β} [ {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - {ωε}_{p}^{0.5} ]$$

$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - β {σ}_{p}}{ω} ]}^{2}$$

Preferably, when the rock expansive stress does not exist, the rock expansive strain is:

$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}}{ω} ]}^{2}$$

when the rock expansive strain does not exist, the rock expansive stress is:

$${σ}_{p} = \frac{{k}_{r} {ρ}_{d}}{β} {( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}$$

Compared with the prior art, the beneficial effects of the present invention are as follows:

(1) The present invention introduces parameters comprising category, density, and water content that affect the rock expansive properties into the analysis of rock expansive stress and rock expansive strain, which can truly reflect the variation of rock expansive stress and rock expansive strain, and can achieve quantized calculation of the rock expansive state.

(2) The present invention introduces rock expansive capacity into quantized calculation of rock expansive properties, which provides a reference index for evaluating the rock expansive properties.

(3) The present invention determines a method for analyzing the constitutive relationship of confined rock expansion, thereby providing a theoretical basis for calculating and judging rock expansive behavior in actual engineering.

### BRIEF DESCRIPTION OF THE DRAWINGS

The drawings described herein provide a further understanding of the present invention, and form a part of the present application. The schematic embodiments of the present invention and the description thereof are used to explain the present invention, and do not limit the present invention. In the figures:

FIG. 1 is a flowchart according to an embodiment of the present invention;

FIG. 2 is a schematic diagram of the relationship between rock expansive capacity and water absorption rate;

FIG. 3 is a schematic diagram of the relationship between rock expansive stress and water content;

FIG. 4 is a schematic diagram of the relationship between rock expansive stress and initial water content;

FIG. 5 is a schematic diagram of the relationship between rock expansive strain and water content;

FIG. 6 is a schematic diagram of the relationship between rock expansive strain and initial water content;

FIG. 7 is a schematic diagram of the relationship between rock expansive strain and rock expansive stress.

### DETAILED DESCRIPTION OF EMBODIMENTS

Embodiments of the technical solutions of the present invention will be described in detail below with reference to the accompanying drawings. The following embodiments are only used to illustrate the technical solutions of the present invention more clearly, and are therefore only used as embodiments, and cannot be used to limit the protection scope of the present invention.

### Embodiment 1

As shown in FIG. 1, a method for analyzing the constitutive relationship of confined rock expansion includes the following steps:

Step 1, acquiring parameters that affect rock expansive properties through experiments, wherein the parameters comprise category, density, and water content. the parameters of category and density can be acquired by mechanical experiments, and water content can be acquired by water content test. It is necessary to acquire the following water content respectively: \textcircled{1} water content in rock expansive initial state; \textcircled{2} water content in rock expansive state; \textcircled{3} maximum water content of the rock, which means the water content when the rock expansive behavior reaches the limit state; \textcircled{4} water content at the point when the water absorption rate changes from rapid increase to slow increase in the rock expansive process.

In the rock expansive process, water content is a critical factor affecting expansion, and rock density has varying degree of influence on both water absorption rate and rock expansive capacity. Therefore, it is particularly important to establish the constitutive relationship of density, water content and rock expansive capacity among various rock parameters. It is the step \textcircled{2} that establishing an expression of the relationship among the rock density, water content and rock expansive capacity.

Rock expansive stress and rock expansive strain generated after rock expansion are related to the rock expansive capacity. Therefore, it is the step \textcircled{3} that establishing an expression of the relationship among the rock expansive capacity, rock expansive stress and rock expansive strain in external expression.

The parameter of rock is a major influencing factor of the rock expansive capacity, especially a change in water content. While the rock expansive stress and the rock expansive strain influenced by the confinement is a negative factor of the rock expansive capacity. Therefore, it is the step \textcircled{4} that obtaining the relationship among rock parameters, rock expansive stress and rock expansive strain according to the rock expansive capacity combined with its intrinsic parameters and external expression.

In step \textcircled{2}, the relationship between the parameters (category, density, and water content) and the rock expansive capacity is as follows:

$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} ;$$

Wherein, kᵣ is rock expansive active coefficient, m·N/kg, which represents the expansion energy of expansive rock per unit mass and reflects the expansion difference of different rock types; ρ_{d} is rock density, kg/m³; w is water content in rock expansive state; w₀ is water content in rock expansive initial state; wₘₐₓ is maximum water content, which means the water content when the rock expansive behavior reaches the limit state; wᵥ is water content at the point when the water absorption rate changes from rapid increase to slow increase in the rock expansive process, wᵥ can be taken as an average value of the water content in rock expansive initial state and the maximum water content.

In step \textcircled{3}, the magnitude of rock expansive stress and rock expansive strain depends on the rock expansive capacity corresponding thereto, the rock expansive capacity corresponding to rock expansive stress is independent from the rock expansive capacity corresponding to rock expansive strain, the sum of the rock expansive capacity Q_{σ} corresponding to the rock expansive stress and the rock expansive capacity Q_{ε} corresponding to the rock expansive strain are equal to the total rock expansive capacity Qₛ, and the expression of their relationship is:

Qₛ=Q_{σ}+Q_{ε};

wherein, Q_{σ} is rock expansive capacity corresponding to the rock expansive stress in the rock expansive behavior, and Q_{ε} is rock expansive capacity corresponding to the rock expansive strain in the rock expansive behavior.

The relationship between the rock expansive capacity Q_{σ} and its corresponding the rock expansive stress σₚ is as follows:

Q_{σ}=βσₚ;

wherein, β is conversion coefficient of the rock expansive stress.

The relationship between the rock expansive capacity Q_{ε} and its corresponding rock expansive strain εₚ is as follows:

Q_{ε}=ωεₚ^{0.5}

wherein, ω is conversion coefficient of the rock expansive strain, kPa.

The conversion coefficient β between the rock expansive stress and its corresponding rock expansive capacity, and the conversion coefficient ω between the rock expansive strain and its corresponding rock expansive capacity vary with the category, density, and water content of rock.

That is:

Qₛ=βσₚ+ωεₚ^{0.5};

In step \textcircled{4}, the rock expansive capacity can be expressed as follows:

$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} = {Q}_{σ} + {Q}_{ε} ;$$

$${Q}_{s} = {βσ}_{p} + {ωε}_{p}^{0.5} ;$$

Thus, the relationship among rock parameters, rock expansive stress and rock expansive strain can be expressed as:

$${σ}_{p} = \frac{1}{β} [ {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - {ωε}_{p}^{0.5} ]$$

$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - {βσ}_{p}}{ω} ]}^{2}$$

The conversion coefficient β of the rock expansive stress and the conversion coefficient ω of rock expansive strain are acquired by fitting the rock expansive stress-strain test data curve under a certain water absorption rate condition by the rock expansive stress-strain expression.

There are three external expressions in the rock expansive process: single expansive stress; single expansive strain; expansive stress and expansive strain occur simultaneously. In the expression of the relationship among rock parameters, rock expansive stress and rock expansive strain, when σ=0, it is corresponded to single rock expansive strain state in confined rock expansion, which is expressed as follows:

$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}}{ω} ]}^{2}$$

when εₚ=0, it is corresponded to single rock expansive stress state in confined rock expansion, which is expressed as follows:

$${σ}_{p} = \frac{{k}_{r} {ρ}_{d}}{β} {( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}$$

### Embodiment 2

Taking gypsum expansive rock as an example, the experimental parameters of the gypsum expansive rock are: the maximum water content wₘₐₓ is 24%, the water content in rock expansive initial state w₀ is 0, the water content at the point when the water absorption rate changes from rapid increase to slow increase in the rock expansive process wᵥ is 12%, the rock expansive active coefficient kᵣ is 3×10⁻⁵ m·N/kg, the rock density ρ_{d} is 2500 kg/m³, the conversion coefficient of the rock expansive stress β is 0.4, and the conversion coefficient of the rock expansive strain ω is 90 kPa.

(1) Establishing the relationship between rock expansive capacity and water absorption rate.

By substituting w₀=0, wᵥ=12%, kᵣ=3×10⁻⁵ m·N/kg, ρ_{d}=2500 kg/m³ into the expression

$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}$$

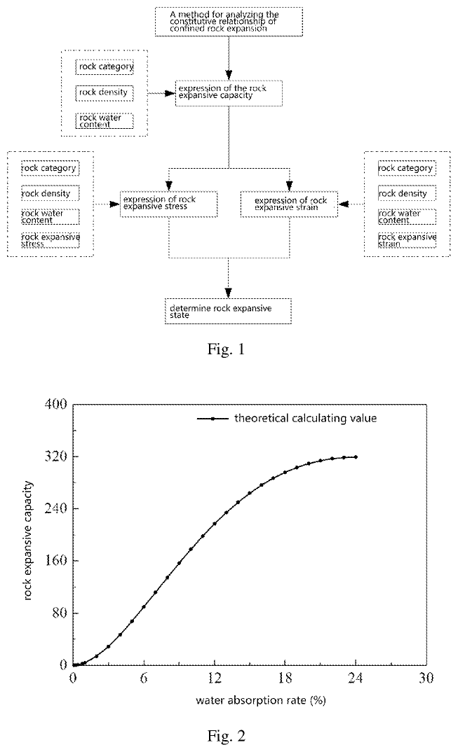

to obtain a change curve between the rock expansive capacity and the water absorption rate, as shown in FIG. 2.

(2) Establishing the relationship between rock expansive stress and water content.

By substituting w₀=0 and 4%, wᵥ=12%, kᵣ=3×10⁻⁵ m·N/kg, ρ_{d}=2500 kg/m³ into the expression

$${σ}_{p} = \frac{{k}_{r} {ρ}_{d}}{β} {( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}$$

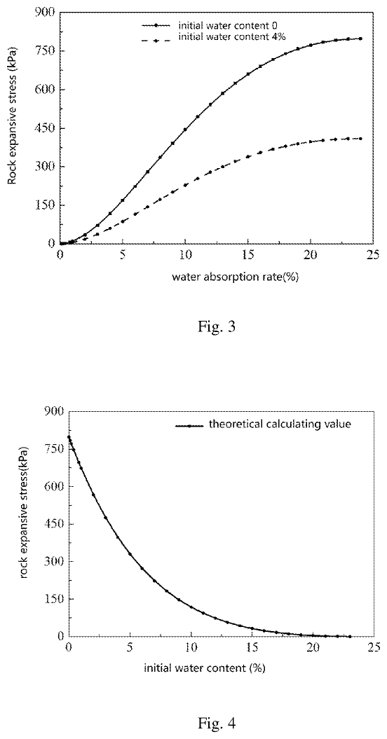

to obtain a change curve between rock expansive stress and water content, as shown in FIG. 3.

(3) Establishing the relationship between rock expansive stress and initial water content.

By substituting wₘₐₓ=24%, wᵥ=12%, kᵣ=3×10⁻⁵ m·N/kg, ρ_{d}=2500 kg/m³ into the expression

$${σ}_{p} = \frac{{k}_{r} {ρ}_{d}}{β} {( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}$$

to obtain a change curve between rock expansive stress and initial water content, as shown in FIG. 4.

(4) Establishing the relationship between rock expansive strain and water content.

By substituting w₀=0 and 4%, wᵥ=12%, kᵣ=3×10⁻⁵ m·N/kg, ρ_{d}=2500 kg/m³ into the expression

$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}}{ω} ]}^{2}$$

to obtain a change curve between rock expansive strain and water absorption rate, as shown in FIG. 5.

(5) Establishing the relationship between rock expansive strain and initial water content in rock expansive initial state.

By substituting wₘₐₓ=24%, wᵥ=12%, kᵣ=3×10⁻⁵ m·N/kg, ρ_{d}=2500 kg/m³ into the expression

$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}}{ω} ]}^{2}$$

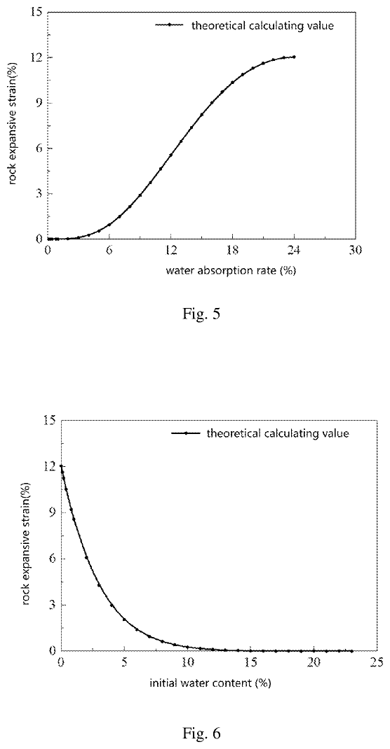

to obtain a change curve between rock expansive strain and initial water content, as shown in FIG. 6.

(6) Establishing the relationship between rock expansive strain and rock expansive stress.

By substituting w₀=0, wᵥ=12%, wₘₐₓ=24%, kᵣ=3×10⁻⁵ m·N/kg, ρ_{d}=2500 kg/m³ into the expression

$${σ}_{p} = \frac{1}{β} [ {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - {ωε}_{p}^{0.5} ]$$

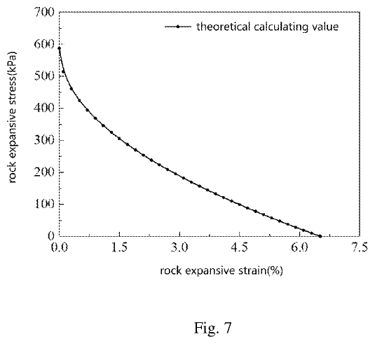

to obtain a change curve between rock expansive strain and rock expansive stress, as shown in FIG. 7.

Based on the description of the above embodiments, the relationship among parameters comprising category, density and water content, rock expansive stress and rock expansive strain can be acquired, thus providing a method for analyzing the constitutive relationship of confined rock expansion.

The above description is merely a preferred embodiment of the present invention, and is not intended to limit the present invention. For the person skilled in the art, various changes and variations can be made to the present invention. Any modifications, equivalent replacements and improvements made within the spirit and principle of the present invention; all should be within the scope of protection of the present invention.

I/We claim:

1. A method for analyzing the constitutive relationship of confined rock expansion, comprising:
(1) determining parameters that affect rock expansive properties, wherein the parameters comprise category, density, and water content;
(2) establishing an expression of the relationship between the parameters and rock expansive capacity;
(3) establishing an expression of the relationship among the rock expansive capacity, rock expansive stress and rock expansive strain; and
(4) determining an expression of the relationship among the parameters, the rock expansive stress and the rock expansive strain according to the rock expansive capacity.

2. The method of claim 1, wherein in step (2), the relationship between the parameters and the rock expansive capacity is as follows:
$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} ;$$
wherein,
Qₛ is the rock expansive capacity,
kᵣ is rock expansive active coefficient, m·N/kg,
ρ_{d} is rock density, kg/m³,
w is water content in rock expansive state,
w₀ is water content in rock expansive initial state,
wₘₐₓ is water content in rock expansion reaching limit state,
wᵥ is water content at the point when the water absorption rate changes from rapid increase to slow increase in rock expansive process.

3. The method of claim 1, wherein in step (3), the relationship among the rock expansive capacity, the rock expansive stress and the rock expansive strain is as follows:
Qₛ=Q+Q_{ε};
wherein,
Qₛ is the rock expansive capacity,
Q_{σ} is rock expansive capacity corresponding to the rock expansive stress, and
Q_{ε} is rock expansive capacity corresponding to the rock expansive strain;
the relationship between the rock expansive stress σₚ and its corresponding rock expansive capacity Q_{σ} is as follows:
Q_{σ}=βσₚ;
wherein,
β is conversion coefficient of the rock expansive stress;
the relationship between the rock expansive strain εₚ and its corresponding rock expansive capacity Q_{ε} is as follows:
Q_{ε}=ωεₚ^{0.5};
wherein,
ω is conversion coefficient of the rock expansive strain, kPa.

4. The method of claim 1, wherein in step (4), the rock expansive capacity is:
$${Q}_{s} = {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} ;$$
$${Q}_{s} = {Q}_{σ} + {Q}_{ε} = {βσ}_{p} + {ωε}_{p}^{0.5} ;$$
wherein,
Qₛ is the rock expansive capacity,
kᵣ is rock expansive active coefficient, m·N/kg,
ρ_{d} is rock density, kg/m³,
w is water content in rock expansive state,
w₀ is water content in rock expansive initial state,
wₘₐₓ is water content in rock expansion reaching limit state,
wᵥ is water content at the point when the water absorption rate changes from rapid increase to slow increase in rock expansive process,
Q_{σ} is rock expansive capacity corresponding to the rock expansive stress,
Q_{ε} is rock expansive capacity corresponding to the rock expansive strain,
β is conversion coefficient of the rock expansive stress,
σₚ is the rock expansive stress,
ω is conversion coefficient of the rock expansive strain,
εₚ is the rock expansive strain;
the relationship among the parameters, the rock expansive stress and the rock expansive strain is as follows:
$${σ}_{p} = \frac{1}{β} [ {k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - {ωε}_{p}^{0.5} ] ;$$
$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} - {βσ}_{p}}{ω} ]}^{2} .$$

5. The method of claim 4, when the rock expansive stress does not exist, the rock expansive strain is:
$${ε}_{p} = {[ \frac{{k}_{r} {{ρ}_{d} ( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}}}{ω} ]}^{2} ;$$
when the rock expansive strain does not exist, the rock expansive stress is:
$${σ}_{p} = \frac{{k}_{r} {ρ}_{d}}{β} {( w - {w}_{0} )}^{2} {e}^{\frac{{w}_{max}}{{w}_{v}} - \frac{w}{{w}_{v}}} {e}^{\frac{{w}_{max}}{{w}_{v}} - \frac{{w}_{0}}{{w}_{v}}} .$$
